(19) United States
(12) Patent Application Publication
(10) Pub. No.: US 2008/0095850 A1
(43) Pub. Date: Apr. 24, 2008

(54) Process for Granulating Particles

(76) Inventors: Jennifer S. Ho, Philadelphia, PA (US); Hubert Dumont, Beaconsfield (CA); Christopher Mancinelli, Collegeville, PA (US); Suhas D. Shelukar, Lansdale, PA (US)

Correspondence Address: MERCK AND CO., INC, P O BOX 2000, RAHWAY, NJ, 07065-0907, US

(21) Appl. No.: 11/665,319

(22) PCT Filed: Oct. 31, 2005
(86) PCT No.: PCT/US05/39238

§ 371 Date: Apr. 13, 2007

Related U.S. Application Data
(60) Provisional application No. 60/624,977, filed on Nov. 4, 2004.

(51) Int. Cl. A61K 31/444; A61K 9/14; A61P 29/00

(52) U.S. Cl. 424/489; 514/334

(57) ABSTRACT

The invention encompasses a process for granulating particles that produces homogeneous, free flowing, attrition resistant, uniform sized granules. When utilized with active pharmaceutical ingredients, such granules can be further processed into controlled released or taste-masked pharmaceutical formulations. Particularly, the process can be utilized to make an oral granule formulation of etoricoxib for treating pain and inflammation in patients that cannot swallow a tablet, such as young children and the elderly.

### BACKGROUND OF THE INVENTION

The Wurster unit operation is commonly used for applying a layer of coating over a substrate in the pharmaceutical industry. The Wurster unit consists of two concentric cylinders, the insert and the annulus, above a distributor plate. The solids to be coated are loaded in the annulus. Upon initiation of airflow, the solids from the annulus pass through the partition gap and are pneumatically conveyed into the insert. The coating solution is sprayed through the nozzle at the distributor plate and coats the solids flowing in the insert. The solids lose their momentum in the fountain zone and fall back into the annulus where they move downward and back into the insert. The deposited coat dries mainly in the insert and fountain zone. The recirculation is continued until the desired coat weight is achieved.

The present invention is directed to a Wurster granulation process, which is a process for granulating pharmaceutical ingredients using a Wurster unit operated above the mass transfer limit. The invention also encompasses a one-step process that encompasses granulation and coating for the preparation of taste-masked or controlled release API formulations using the Wurster granulation process. The invention also allows for the granulation of materials of different physical characteristics using the Wurster granulation process, e.g., beads/agglomerates/granules (mean less than 300 μm) with powders (mean less than 150 μm).

The Wurster granulation process has distinct advantages over the conventional high-shear and fluid-bed granulation processes. These advantages over the conventional granulation process are:

(1) The recirculation in the Wurster granulation process provides uniform distribution of the granulating solution to the solid particles, resulting in uniform and homogeneous granulation. The distribution of granulating solution onto the solid particles in the high-shear and fluid-bed granulation processes is restricted to the event when the solid particles are exposed to the spray zone. This is due to the narrow spray zone as compared to the entire solids bed in the conventional processes. In addition, the exposure of solid particles being exposed to the narrow spray zone is uncontrolled, chaotic in the conventional processes as compared to the ordered recirculation process in the Wurster granulation process. Thus, the Wurster granulation process due to its orderly recirculation imparts uniform granulation characteristics and better control of granulation as compared to that in the conventional granulation processes.

(2) The uniform granulation enables tighter control of the granule size distribution for special applications such as controlled release or taste-masked technology.

(3) The Wurster unit operation allows one step process for taste masking and controlled release applications where the granulation step can be followed by incorporation of taste masking or controlled release coat by conducting coating in the same Wurster unit.

(4) Wurster granulation provides the ability to quantify and scale up the granulation process using chemical engineering principles. The granulation kinetics can be easily related to heat/mass transfer and hydrodynamic characteristics since application of these principles have already been demonstrated for coating processes in the Wurster unit. Geometric scale-up issues can be minimized by utilizing multiple development-scale inserts in the commercial-scale Wurster unit.

(5) Wurster granulation has potential for providing granules with better attrition resistance than the high shear granulation/fluid-bed drying processes since the granules are prepared under high velocity/impact conditions in the Wurster unit operation.

(6) Facilitates on-line control of granule size using existing technology since sticking issues observed in high-shear and fluid-bed granulations are not present in the annulus region of the Wurster unit.

### SUMMARY OF THE INVENTION

The invention encompasses a process for granulating particles that produces homogeneous, free flowing, attrition resistant, uniform sized granules. When utilized with active pharmaceutical ingredients, such granules can be further processed into controlled released or taste-masked pharmaceutical formulations. Particularly, the process could be utilized to make an oral granule formulation of etoricoxib for treating pain and inflammation in patients that cannot swallow a tablet, such as young children and the elderly.

### BRIEF DESCRIPTION OF THE DRAWINGS

FIG. 1. Schematic of Wurster unit operation.

FIG. 2. Particle size distribution of etoricoxib pediatric formulation.

FIG. 3. Particle size distributions of placebo pediatric formulation showing the consolidation and growth of ingredients with time.

FIG. 4. Particle size distribution of etoricoxib pediatric low dose formulation.

FIG. 5. Particle size distributions for the Wurster granulation of aprepitant showing particle growth with time.

### DETAILED DESCRIPTION OF THE INVENTION

The invention encompasses a process for granulating particles by subjecting the particles to a repeated circulating movement comprising:

(a) a non-rotating upward pneumatical movement from a starting area inside a vertical granulation pipe, wherein said particles are subjected to a spray of droplets of granulation solution, and

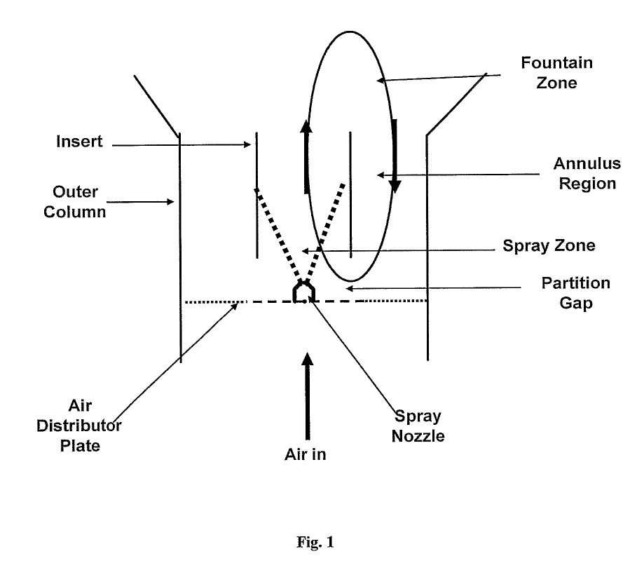

(b) a downward movement outside said pipe and a horizontal movement towards the starting area for said pneumatical movement,

wherein said process is operated above the mass transfer limit to facilitate the agglomeration of the particles. This process is exemplified in FIG. 1.

An embodiment of the invention encompasses the above process wherein a portion or all of the particles comprise an active pharmaceutical ingredient.

Another embodiment of the invention encompasses the above process wherein the active pharmaceutical ingredient is an anti-inflammatory agent. Within this embodiment the active pharmaceutical ingredient is etoricoxib. Also within this embodiment, the particles comprising etoricoxib are microspheres.

Another embodiment of the invention encompasses the above process wherein the granules produced by the process are further processed into a tablet, capsule or oral granules.

Another embodiment of the invention encompasses the above process wherein the granules produced by the process are further processed into a controlled release formulation.

Another embodiment of the invention encompasses the above process wherein the granules produced by said process are further processed into an oral granule formulation. Within this embodiment, the granulation solution comprises at least one of the following ingredients:

(a) a taste-masking agent,

(b) a sweetening agent, and

(c) a flavoring agent,

and optionally a binder.

In another embodiment, the taste-masking agent is selected from the group consisting of: polymethacrylate, hydropropylmethylcellulose, hydroxypropylcellulose and vinyl pyrrolidone-vinyl acetate co-polymer.

In another embodiment, the sweetening agent is selected from the group consisting of: sugar and aspartame.

In another embodiment, the flavoring agent is artificial cherry flavor.

Another embodiment of the invention encompasses the above process wherein the particles comprising the active pharmaceutical ingredient are blended with particles that function as a bulking agent prior to granulation. Within this embodiment, the particles that function as a bulking agent are selected from the group consisting of: mannitol, lactose, starch and calcium phosphate.

In another embodiment, the invention encompasses the above process wherein the active pharmaceutical ingredient is etoricoxib and the particles comprising etoricoxib are blended with mannitol prior to granulation and wherein the granulation solution comprises hydroxypropyl cellulose, artificial cherry flavor and aspartame. In this embodiment, the volume mean diameter of the final product is about 800 μm. The invention also encompasses a pharmaceutical composition comprising granules produced by this process.

In another embodiment the invention encompasses an oral granule pharmaceutical composition comprising:

(1) about 1 to about 39% wt/wt of a plurality of coated etoricoxib microspheres, said microspheres comprising about 19% wt/wt of etoricoxib, about 46% wt/wt distilled monoglyceride 03-VF, about 12% wt/wt milled Gelucire 50/13, about 9% wt/wt Eudragit® NE30D, about 2% wt/wt Methocel and about 12% wt/wt microtalc 1538; with the remainder up to 39% wt/wt comprising a plurality of sugar spheres;

(2) about 50% wt/wt of mannitol; and

(3) a coating and binding solution comprising about 8% wt/wt hydroxypropyl cellulose, about 3% wt/wt artificial cherry flavor and about 1% wt/wt aspartame.

Within this embodiment, the invention encompasses the oral granule pharmaceutical composition described above selected from the group consisting of:

(A) about 13% wt/wt of a plurality of coated etoricoxib micropsheres and about 26% of a plurality of sugar spheres;

(B) about 16% wt/wt of a plurality of coated etoricoxib micropsheres and about 23% of a plurality of sugar spheres;

(C) about 21% wt/wt of a plurality of coated etoricoxib micropsheres and about 18% of a plurality of sugar spheres; and

(D) about 26% wt/wt of a plurality of coated etoricoxib micropsheres and about 13% of a plurality of sugar spheres.

The term “coated etoricoxib microsphere” above is described in Preparative Example 1.

The term “non-rotating” means that the particles are not subjected to a swirling flow of drying and carrying gas which would cause the particles to flow in a rotational-symmetrical pattern as described in U.S. Pat. No. 6,492,024, granted on Dec. 10, 2002.

“API” means active pharmaceutical ingredient.

Etoricoxib is a selective inhibitor of cyclooxygenase-2 which is useful to treat inflammation and pain in a variety of conditions. Etoricoxib is commercially available and sold under the trade name ARCOXIA (Merck & Co., Inc.) Etoricoxib is taught in U.S. Pat. No. 5,861,419, granted on Jan. 19, 1999. Methods for making etoricoxib are taught in U.S. Pat. No. 6,040,319, granted on Mar. 21, 2000. Etoricoxib is also known by the designation MK-663 Aprepitant is commercially available and sold under the trade name EMEND (Merck & Co., Inc.). Aprepitant is also known by the designation MK-869.

The term “taste-masking agent” means, for example, polymethacrylate (EUDRAGIT), hydropropylmethylcellulose (HMPC), Hydroxypropylcellulose, (HPC) and vinyl pyrrolidone-vinyl acetate co-polymer (PLASDONE).

The term “sweetening agent” means, for example, sugar and aspartame.

The term “flavoring agent” means for example artificial flavor, such as artificial cherry flavor.

The term “bulking agent” means, for example, mannitol, lactose, starch and calcium phosphate.

The term “binder” means, for example, hydroxypropyl cellulose (HPC) or hydroxypropyl methyl cellulose (HPMC).

The term “granulation solution” means, for example, aqueous solution of “binder” agents as defined above.

For coating applications, the process must be operated below the mass transfer limit, which is defined as the operating conditions above which agglomeration of particles takes place. Thus, operating above the mass transfer limit would facilitate agglomeration of particles. The operation of the Wurster granulation is described below. The coating solution is mainly granulating solution with or without binder. The mass transfer limit is characterized by the exhaust relative humidity, exhaust temperature, bed temperature in the annulus and can be controlled by the inlet air flow rate, inlet air temperature, inlet air dew point, granulation solution spray rate, atomizing air flow rate, atomizing air dew point, atomizing air temperature. Thus, the mass transfer limit can be readily determined by one having ordinary skill in the art and is further exemplified in the examples that follow.

The present invention can be used for:

(1) Granulating API with excipients to form homogeneous, free flowing, attrition resistant, uniform sized granules that can be further processed into solid-dosage forms, such as tablets, capsules, and sprinkles.

(2) Granulating excipients to form homogeneous, free flowing, attrition resistant, uniform sized granules that can be further processed into solid-dosage forms, such as tablets, capsules, and sprinkles.

(3) Granulating API to form drug-rich, homogeneous, free flowing, attrition resistant, uniform sized granules that can be further processed into solid-dosage forms, such as tablets, capsules, and sprinkles.

(4) Preparing taste-masked or controlled release formulations in one step rather than multiple steps. In both applications, the initial step consists of granulating the API and/or excipients by operating in the Wurster coating unit above the mass transfer limit. Following granulation, the resulting granules are coated with a taste mask layer or diffusion-type controlled release coat by operating in the same Wurster coating unit below the mass transfer limit.

(5) Granulating particles of different size and shape, which are otherwise difficult to granulate with other known processes such as high-shear and fluid-bed granulation processes.

The invention is further exemplified in the examples that follow.

### PREPARATORY EXAMPLE 1

### Coated Etoricoxib Microspheres

Coated etoricoxib microspheres are prepared by following the procedures described in U.S. Pat. No. 5,683,720, granted Nov. 4, 1997 and U.S. Pat. No. 5,849,223, granted Dec. 15, 1998, which are hereby incorporated by reference in their entirety. The composition of the coated etoricoxib microspheres comprises:
(1) about 19% wt/wt etoricoxib, about 46% wt/wt distilled monoglyceride 03-VF and about 12% wt/wt milled Gelucire 50/13 (blended prior to liquiflash processing); and
(2) about 9% wt/wt Eudragit® NE30D, about 2% wt/wt Methocel and about 12% wt/wt microtalc 1538 (coating).

### EXAMPLE 1

### Granulation of API-Containing Core Beads with Powder Excipients

### Etoricoxib Pediatric Formulation

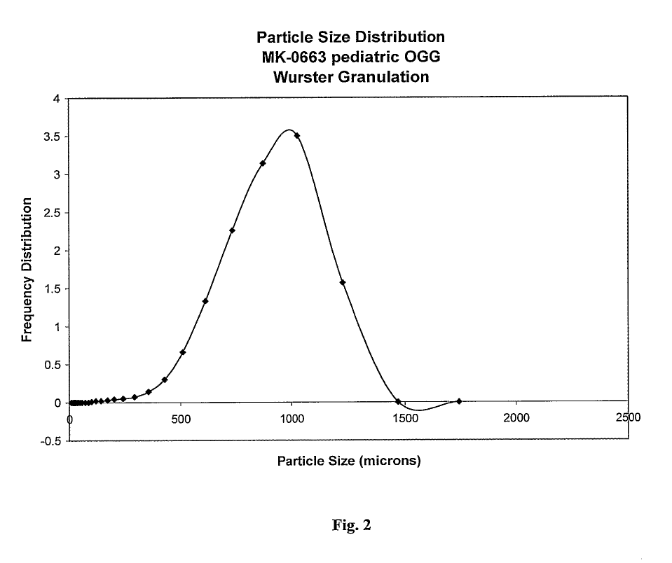

A 1.5 kg batch of etoricoxib oral granules was obtained by granulation in a Glatt GPCG3 GPCG3 fluid bed column equipped with a Wurster insert. A pre-blend containing 585 g of etoricoxib taste-masked microspheres (volume mean diameter of 237 microns) and 742.5 g of mannitol (Pearlitol SD200) (volume mean diameter of 137 microns), was charged into the column and fluidized while a solution of 8% w/w hydroxypropyl cellulose (Klucel LF), 2.5% w/w Artificial Cherry Flavor and 1% w/w Aspartame was sprayed from below into the partition section of the Wurster column. Inlet air flow rate was changed during the progression of the granulation to ensure an adequate particle flow pattern throughout the granulation. The volume mean diameter of the final product was 799 microns as measured by laser diffraction. Scanning electron microscopy of the initial pre-blend and final granulation confirmed the formation of agglomerated etoricoxib microspheres and mannitol particles. The final particle size distribution is shown in FIG. 2.

### EXAMPLE 2

### Granulation of Excipient Beads with Powder Excipients

### Granulation of Pharmaceutical Ingredients with Different Physical Characteristics

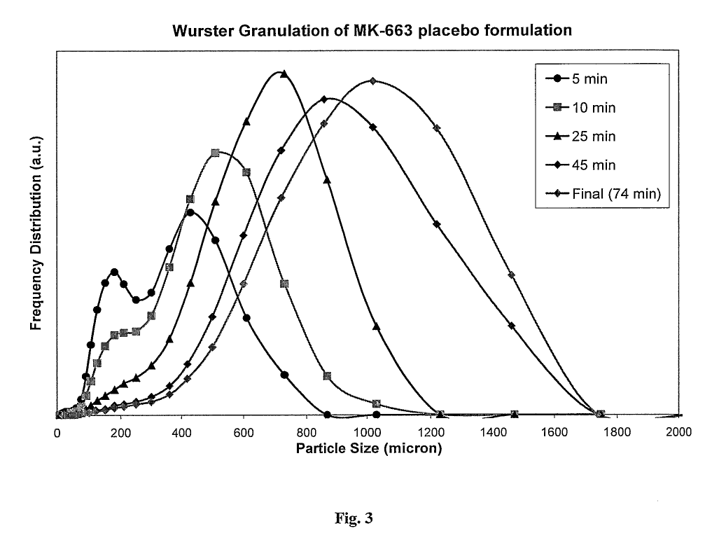

1.5 kg and 10 kg of a granulation containing 39% Sugar spheres (Non-pareil) and 49.5% of mannitol Pearlitol (SD200) were obtained in a Glatt GPCG3GPCG3 and GPCG15GPCG15 fluid bed column respectively. The fluid beds were equipped with Wurster insert and charged with a pre-blend containing sugar spheres 40-60 mesh and of mannitol (Pearlitol SD200) (volume mean diameter of 137 microns), and fluidized while a solution of 8% w/w hydroxypropyl cellulose (Klucel LF), 2.5% w/w Artificial Cherry Flavor and 1% w/w Aspartame was sprayed from below into the partition section of the Wurster column. Inlet air flow rate was changed during the progression of the granulation to ensure an adequate particle flow pattern throughout the granulation. The volume mean diameter of the batch of the final product was between 750 and 800 microns as measured by laser diffraction. Scanning electron microscopy of the initial pre-blend and final granulation confirmed the formation of agglomerated sugar spheres and mannitol particles. The particle size distributions showing the consolidation and growth of ingredients with time is demonstrated in FIG. 3.

### EXAMPLE 3

### Granulation of API-Containing Beads, Excipient Beads, and Powder Excipients

### Granulation of Pharmaceutical Ingredients with Different Physical Characteristics

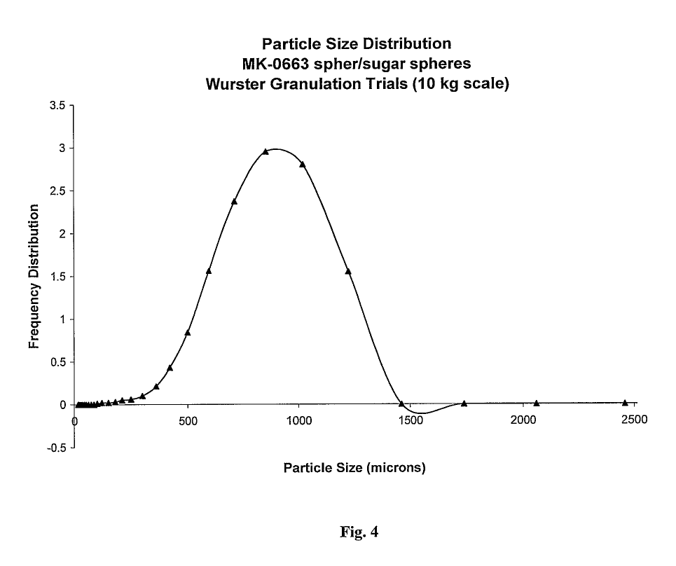

1.5 kg and 10 kg of a granulation containing 13% etoricoxib taste masked microspheres, 26% sugarsugar spheres (Non-pareil) and 49.5% of mannitol (Pearlitol SD200) were obtained in a Glatt GPCG3GPCG3 and GPCG15GPCG15 fluid bed column respectively. The fluid beds were equipped with Wurster insert and charged with a pre-blend containing: etoricoxib microspheres (volume mean diameter of 237 microns), sugar spheres 40-60 mesh and of mannitol (Pearlitol SD200) (volume mean diameter of 137 microns), and fluidized while a solution of 8% w/w hydroxypropyl cellulose (Klucel LF), 2.5% w/w Artificial Cherry Flavor and 1% w/w Aspartame was sprayed from below into the partition section of the Wurster column. Inlet air flow rate was changed during the progression of the granulation to ensure an adequate particle flow pattern throughout the granulation. The volume mean diameter of the batch of the final product was between 750 and 850 microns as measured by laser diffraction. Scanning electron microscopy of the initial pre-blend and final granulation confirmed the formation of agglomerated sugar spheres and mannitol particles. The final particle size distribution is shown in FIG. 4.

### EXAMPLE 4

### Granulation of Excipients

A 2 kg batch of Avicel PH101, lactose (hydrous), and hydroxypropyl cellulose (Klucel EXF) was granulated in a Niro MP1 fluid bed column equipped with a Wurster insert. A pre-blend of 940 g Avicel PH101, 940 g lactose (hydrous) and 120 g hydroxypropyl cellulose (Klucel EXF) was charged into the column and fluidized while pure water was sprayed from below into the partition section of the Wurster column. Inlet air flow rate was changed to ensure an adequate particle flow pattern throughout the granulation. The volume mean diameter of the batch was observed to increase from 192 microns to 290 microns as measured by laser diffraction.

### EXAMPLE 5

### Granulation of API to Form Drug-Rich Granules

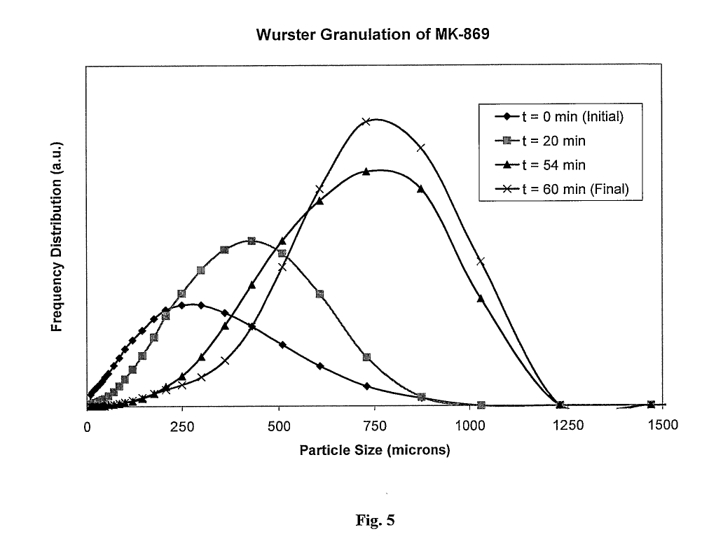

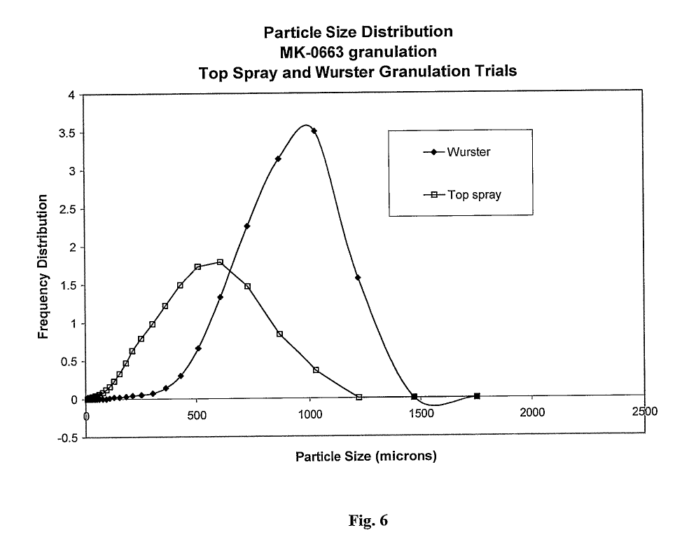

A 1.5 kg batch of aprepitant API was granulated in a Niro MP1 fluid bed column equipped with a Wurster insert. A pre-blend of 1500 g aprepitant was charged into the column and fluidized while a solution of 8% w/w hydroxypropyl cellulose (Klucel EXF) was sprayed from below into the partition section of the Wurster column. Inlet air flow rate was changed to ensure an adequate particle flow pattern throughout the granulation. The volume mean diameter of the batch was observed to increase from 201 microns to 573 microns as measured by laser diffraction. Scanning electron microscopy of the initial pre-blend and final granulation confirmed the formation of agglomerated aprepitant drug particles. The particle size distribution showing particle growth with time is shown in FIG. 5.

### EXAMPLE 6

### Granulation of Excipients

A 0.5 kg batch of Mannitol SD200 and hydroxypropyl cellulose (Klucel LF) was granulated in a Glatt GPCG1GPCG1 fluid bed column equipped with a Wurster insert. A pre-blend of 460 g of mannitol was charged into the column and fluidized while HPC solution was sprayed from below into the partition section of the Wurster column. Inlet air flow rate was changed to ensure an adequate particle flow pattern throughout the granulation. The volume mean diameter of the batch was observed to increase from 137 microns to 380 microns as measured sieve analysis.

Results

The formulation of etoricoxib for pediatric use was initiated using a top-spray fluid bed granulation process by which a binder solution is sprayed from above onto a powder pre-blend that is fluidized in a fluid bed column. This process, however, results in a final granulation containing a high percentage of fine material which in turn results in a non-uniform distribution of drug throughout the product batch. Fine material could not be incorporated in the granulation product and batches made using top-spray granulation failed specifications for blend uniformity. Wurster granulation was introduced as a process to solve this problem. While the top-spray granulation process produced batches with 21-23% of fine material (i.e. <250 microns), Wurster granulation produced batches containing less 4% of fine material. Blend uniformity specifications have been met for each active batch made in process development for the etoricoxib pediatric formulation.

What is claimed is:

1. A process for granulating particles by subjecting the particles to a repeated circulating movement comprising:
(a) a non-rotating upward pneumatical movement from a starting area inside a vertical granulation pipe, wherein said particles are subjected to a spray of droplets of granulation solution, and
(b) a downward movement outside said pipe and a horizontal movement towards the starting area for said pneumatical movement,
wherein said process is operated above the mass transfer limit to facilitate the agglomeration of the particles.

2. The process according to claim 1 wherein a portion or all of the particles comprise an active pharmaceutical ingredient.

3. The process according to claim 2 wherein the active pharmaceutical ingredient is an anti-inflammatory agent.

4. The process according to claim 3 wherein the active pharmaceutical ingredient is etoricoxib.

5. The process according to claim 4 wherein the particles comprising etoricoxib are microspheres.

6. The process according to claim 2 wherein the granules produced by the process are further processed into a tablet, capsule or oral granules.

7. The process according to claim 2 wherein the granules produced by the process are further processed into a controlled release formulation.

8. The process according to claim 2 wherein the granules produced by said process are further processed into an oral granule formulation.

9. The process according to claim 8 wherein the granulation solution comprises at least one of the following ingredients:
(a) a taste-masking agent,
(b) a sweetening agent, and
(c) a flavoring agent,
and optionally a binder.

10. The process according to claim 9 wherein the taste-masking agent is selected from the group consisting of: polymethacrylate, hydropropylmethylcellulose, hydroxypropylcellulose and vinyl pyrrolidone-vinyl acetate co-polymer.

11. The process according to claim 9 wherein the sweetening agent is selected from the group consisting of: sugar and aspartame.

12. The process according to claim 9 wherein the flavoring agent is artificial cherry flavor.

13. The process according to claim 2 wherein the particles comprising the active pharmaceutical ingredient are blended with particles that function as a bulking agent prior to granulation.

14. The process according to claim 13 wherein the particles that function as a bulking agent are selected from the group consisting of: mannitol, sugar spheres, lactose, starch and calcium phosphate.

15. The process according to claim 9 wherein the active pharmaceutical ingredient is etoricoxib and the particles comprising etoricoxib are blended with mannitol prior to granulation and wherein the granulation solution comprises hydroxypropyl cellulose, artificial cherry flavor and aspartame.

16. The process according to claim 15 wherein the volume mean diameter of the final product is about 800 μm.

17. A pharmaceutical composition comprising granules produced by the process according to claim 15.

18. An oral granule pharmaceutical composition comprising:
(1) about 1 to about 39% wt/wt of a plurality of coated etoricoxib microspheres, said microspheres comprising about 19% wt/wt of etoricoxib, about 46% wt/wt distilled monoglyceride 03-VF, about 12% wt/wt milled Gelucire 50/13, about 9% wt/wt Eudragit® NE30D, about 2% wt/wt Methocel and about 12% wt/wt microtalc 1538; with the remainder up to 39% wt/wt comprising a plurality of sugar spheres;
(2) about 50% wt/wt of mannitol; and
(3) a coating and binding solution comprising about 8% wt/wt hydroxypropyl cellulose, about 3% wt/wt artificial cherry flavor and about 1% Wt/wt aspartame.

19. The oral granule pharmaceutical composition according to claim 18 selected from the group consisting of:
(A) about 13% wt/wt of a plurality of coated etoricoxib micropsheres and about 26% of a plurality of sugar spheres;
(B) about 16% wt/wt of a plurality of coated etoricoxib micropsheres and about 23% of a plurality of sugar spheres;
(C) about 21% wt/wt of a plurality of coated etoricoxib micropsheres and about 18% of a plurality of sugar spheres; and
(D) about 26% wt/wt of a plurality of coated etoricoxib micropsheres and about 13% of a plurality of sugar spheres.
